(19) United States
(12) Patent Application Publication
(10) Pub. No.: US 2024/0094801 A1
(43) Pub. Date: Mar. 21, 2024

(54) CONTEXTUAL VIRTUAL REALITY RENDERING AND ADOPTING BIOMARKER ANALYSIS

(71) Applicant: INTERNATIONAL BUSINESS MACHINES CORPORATION, ARMONK, NY (US)

(72) Inventors: Jeremy R. Fox, Georgetown, TX (US); Alexander Reznicek, Troy, NY (US); Bahman Hekmatshoartabari, White Plains, NY (US); Martin G. Keen, Cary, NC (US)

(21) Appl. No.: 17/934,026

(22) Filed: Sep. 21, 2022

(51) Int. Cl. G06F 3/01; G16H 40/63

(52) U.S. Cl. CPC G06F 3/011; G16H 40/63; G06F 2203/011

(57) ABSTRACT

According to one embodiment, a method, computer system, and computer program product for biometric mixed-reality emotional modification is provided. The present invention may include collecting, by a plurality of biosensors, biometric information on a user during a mixed-reality session, wherein the biometric information comprises biomarkers; identifying, by one or more machine learning models, a mental state of the user based on the biometric information; and responsive to determining that the mental state does not match an intended emotion associated with a mixed-reality experience, modifying the mixed-reality experience with one or more virtual content elements.

### BACKGROUND

The present invention relates, generally, to the field of computing, and more particularly to mixed reality.

Mixed reality is a field concerned with merging real and virtual worlds such that physical and digital objects co-exist and interact in real time. Mixed reality does not exclusively take place in either the physical or virtual worlds but is a hybrid of reality and virtual reality; as such, mixed reality describes everything in the reality-virtuality continuum except for the two extremes, namely purely physical environments, and purely virtual environments. Accordingly, mixed reality includes augmented virtuality (AV), augmented reality (AR) and virtual reality (VR). Mixed reality has found practical applications in such areas as remote working, construction, gaming, and military, academic and commercial training.

### SUMMARY

According to one embodiment, a method, computer system, and computer program product for biometric mixed-reality emotional modification is provided. The present invention may include collecting, by a plurality of biosensors, biometric information on a user during a mixed-reality session, wherein the biometric information comprises biomarkers; identifying, by one or more machine learning models, a mental state of the user based on the biometric information; and responsive to determining that the mental state does not match an intended emotion associated with a mixed-reality experience, modifying the mixed-reality experience with one or more virtual content elements.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a biometric emotional MR modifier process according to at least one embodiment;

FIG. 3 is a diagram illustrating an exemplary biosensor for collecting biomarkers, according to at least one embodiment;

FIG. 4 is a diagram illustrating an exemplary biosensor array for collecting biomarkers, according to at least one embodiment; and

FIG. 5 is a diagram illustrating an exemplary biosensor array for collecting biomarkers, according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to mixed reality. The following described exemplary embodiments provide a system, method, and program product to, among other things, gather biometric information on a user including biomarkers in the user's sweat, utilize the biometric information to identify a mental state of the user, and modify the user's mixed reality experience based on the identified mental state.

As previously described, mixed reality is a field concerned with merging real and virtual worlds such that physical and digital objects co-exist and interact in real time. Mixed-reality systems use software to generate images, sounds, haptic feedback, and other sensations to augment a real-world environment. While the creation of this augmented environment can be achieved with mobile devices such as cell phones or tablets, more specialized equipment is also used, typically in the form of glasses or headsets where computer generated elements are overlaid onto a view of the real world by being projected or mapped onto a lens in front of a user's eyes. With the help of computer augmentation, information about the surrounding world of the user, as well as other digital elements overlaid onto the world, become interactive and digitally manipulable.

Mixed-reality environments may be utilized to immerse a user in environments authored with the intention of invoking particular emotional responses, such as a training simulation, a game, a narrative experience such as a movie, a social platform, et cetera. Authors may utilize a technique called contextual mixed reality, which may be a method of improving the realism of mixed-reality environments by adjusting the mixed-reality environment based on the current emotional state of the user experiencing the mixed-reality environment. In other words, contextual mixed-reality rendering may be the automatic adjustment of environmental factors in a virtual reality based on biometric data gathered from the user that provides insight into the user's emotional state, where biometric data may be measurements of the biological characteristics and/or behavioral characteristics of the user. The aim is to enhance the mixed reality environment to improve the immersion of the user by guiding the user towards the emotions that the mixed-reality experience is intended to evoke, and/or to guide the emotions felt by the user in the mixed reality environment in order to create a more immersive and/or more emotionally pleasant experience for the user, potentially regardless of the emotion the mixed-reality experience is designed to evoke.

One potential application of contextual mixed-reality rendering may include the measurement of stress biomarkers to characterize the emotional state of the user. Stress biomarkers are biological markers that may be used to measure the level of stress a person is experiencing. Stress biomarkers can be measured in a number of ways, including through the use of blood tests, saliva tests, and urine tests; however, such tests necessitate impermissively invasive collection methods and, potentially, trained human personnel. However, collection of sweat is another possible way to measure stress biomarkers, as sweat contains many of the same stress biomarker molecules that are found in blood, saliva, and urine, such as cortisol, dopamine, and neuropeptide Y. Sweat can be collected through a small form-factor biosensor that is non-invasive and requires no trained personnel. Detecting emotions based on biomarkers is the subject of active research, and at present there is no mature technology that can reliably classify emotions based on biomarkers alone. Generally speaking, this is because of two reasons: (i) in order for a biosensor device such as an ion-sensitive transistor to selectively respond to a target biomarker, i.e. sense the presence and concentration of a target biomarker without responding to non-target biomarkers, the sensing surface of the biosensor device needs to be functionalized with a proper chemistry; however, this is difficult and in practice a sensing surface functionalized for a target biomarker produces a measurable response to non-target biomarkers as well, and (ii) a biomarker may correlate to more than one emotion, and more than one biomarker may correlate to an emotion; and perhaps with the exception of stress, there are no well-established rules that can exhaustively correlate emotions with biomarkers.

As such, it may be advantageous to, among other things, implement a system that employs an array of densely packed cross-reactive biosensor devices where the sensing surfaces of the biosensor devices are functionalized differently, allowing the biosensor devices to produce correlated responses to different biomarkers; the system may feed sensor responses from the biosensor devices into a neural network to classify the emotions based on the collective response of the biosensor array. It may further be advantageous to implement a system that uses the sensing of body temperature and speech emotion recognition as additional modalities to improve the overall emotion classification accuracy by using the weighted averages of the different modalities or end-to-end classification with a manager network. It may further be advantageous to implement a system that identifies mixed reality content that matches and/or complements the user's emotional state and modify the mixed reality environment to align the mixed reality environment with the user's emotional state.

The present embodiment has the capacity to improve the technical field of mixed reality by improving the accuracy of emotional characterization by using an array of differently functionalized biosensors sensing biomarkers in sweat, as well as body temperature and speech recognition, to feed a neural network which classifies emotions based on the collective response of the biosensor array rather than individual responses of differently-functionalized biosensors; this further eliminates non-ideal biosensor selectivity as an issue since the neural network classification is based on the collective response patterns rather than individual biosensor responses. Furthermore, a neural network can learn complex correlations/response patterns only by observing the data and does not need to be provided any correlation rules during training, reducing the complexity of deploying the system. Additionally, this improved accuracy of emotional characterization enables embodiments of the invention to improve the alignment of a mixed reality environment to a user's emotions to increase the user's immersion in the mixed reality environment, and to thereby improve the user's mixed-reality experience.

According to at least one embodiment, the invention is a method of recording biometric information of the user during a mixed reality session, measuring an emotional state of a user during the mixed reality session based on the recorded biometric information, identifying mixed reality content that matches the measured emotional state based on a profile, and modifying the mixed-reality session with the identified mixed-reality content.

According to at least one embodiment, the mixed reality session may be a discrete period of time where the user is interacting with a particular mixed reality program or experience through a mixed reality device. The mixed reality program may be a software program such as a training simulator, a game, a narrative experience such as a movie, a social platform, et cetera that creates or provides one or more mixed-reality experiences for the user to interact with through a mixed reality device. The mixed reality experiences may be discrete episodes comprising one or more scenes, virtual objects, narrative elements, graphical elements, simulated characters, et cetera; the mixed reality experiences may include training simulations, virtual tours, story vignettes, chapters of a game, et cetera. The entire mixed reality program, and/or portions of it such as important story beats, may be associated with a specific emotion, such as joy, fear, stress, anger, et cetera, which the mixed reality program is intended to evoke in a user. For example, a mixed reality experience comprising a petting zoo may be intended to evoke joy, and not stress, anger, or fear, whereas the climax to a horror experience may be intended to evoke fear and stress.

The mixed reality device may be any device or combination of devices enabled to record real-world information that the mixed reality program may overlay with computer-generated perceptual elements to create the mixed-reality reality environment; the mixed reality device may further record the actions, position, movements, et cetera of the user, to track the user's movement within and interactions with the mixed reality environment. The mixed reality device may display the mixed reality environment to the user. The mixed reality device may be equipped with or comprise a number of sensors such as a camera, microphone, accelerometer, et cetera, and these sensors and/or may be equipped with or comprise a number of user interface devices such as displays, touchscreens, speakers, et cetera. One or more of the sensors may be capable of capturing biometric data and may accordingly be herein referred to as biometric sensors or biosensors. In some embodiments, the mixed reality device may be a headset that is worn by the viewer.

Biometric data of the user may be data pertaining to unique physical or behavioral characteristics of the user, such as pupil width, eye movement, stress responses, heart rate, breathing rate, speech, body temperature, et cetera. Biometric information, as referred to herein, may also encompass the position and movements of the user, as well as sounds and speech of the user relevant to determining mental state. Biometric information may be collected from the user only after obtaining a user's express permission. Biometric information may be collected by one or more sensors such as pupillometry sensors, infrared and visible light cameras, heart rate monitors, blood pressure monitors, et cetera, and may be standalone or may be integrated into devices such as wearable vitality trackers, mixed reality devices, mobile devices, et cetera. Biometric information of a user may be collected continuously or at regular intervals during a mixed reality session. In some embodiments of the invention, biometric information may be collected at or immediately prior to points indicated by an author of a mixed-reality experience where alignments of the mixed reality environment with the user's emotional state may be most impactful, for example story beats including the climax, action scenes, emotional dialogue scenes, et cetera.

Biometric data may include data regarding the presence of stress biomarkers in the user's sweat. Stress biomarkers are biological markers that may be used to measure the level of stress an individual such as a user is experiencing. Stress biomarkers can be measured within sweat, as sweat contains many of the same stress biomarker molecules that are found in blood, saliva, and urine, such as cortisol, dopamine, and neuropeptide Y. Cortisol is a steroid hormone that is released in response to stress which increases blood sugar, blood pressure, and prepares the body to fight or flee by increasing heart rate and blood flow to the muscles, and which also suppresses the immune system. Dopamine is a neurotransmitter that is involved in addiction, pleasure seeking, fear, and stress. Neuropeptide Y is a peptide that is released by the hypothalamus in response to stress, and which increases heart rate, blood pressure, and food intake, and stimulates the release of cortisol.

According to at least one embodiment, the emotional state or mental state of a user may be the dominant emotion including anger, calmness, sadness, stress, anxiety, boredom, fright, et cetera experienced by a user at any given point during a mixed-reality session. The emotional state of a user may be assessed based on biometric data of the user. In some embodiments, the mental state may comprise multiple emotions, such as when the user is equally feeling both joy and sadness, for example at the conclusion of a mixed-reality narrative experience. Biometric information is not the user's emotional state, but elements of biometric information may individually or in combination indicate an emotional state in the user. For example, a high heart rate and/or the presence of stress biomarkers above a threshold concentration in the user's sweat indicates stress; a high heart rate, stress biomarkers above a threshold concentration, and a smile or cheerful words or paralinguistic vocalizations may indicate excitement/joy, and a high heart rate, stress biomarkers above a threshold concentration, and harsh shouting or violent motion may indicate anger.

In some embodiments of the invention, biometric sensors for sensing biomarkers in sweat may be organized into a monolithically integrated array of two or more biometric sensors. Each biometric sensor may be functionalized by treating its sensing surface for targeting one or more biomarkers in sweat. The materials and approaches known in the art for functionalization of the sensing surfaces may be used. The sensor array may include redundancy for improving fault tolerance, facilitating anomaly detection by machine learning algorithms, and improving SNR by averaging out uncorrelated noise. The peripheral/driver circuitry as well as any additional circuitry/functionality may be integrated monolithically on the sensor chip. In some embodiments, the sensor chip may be bonded, for example using flip-chip bonding, to a peripheral chip including further functionality such as memory, battery, communication, processing, et cetera. Moreover, the sensor chip, with or without a bonded chip, may be integrated heterogeneously, for example with separately manufactured components, into a higher-level assembly, such as a System-in-Package (SIP). The SIP may be, for example, a handheld/portable device such as a phone, tablet, or wearable mixed-reality device. In some embodiments, the sensor chip may be transferred onto a flexible substrate, allowing the sensor array to be worn like a patch. Prior to operation, the sensor arrays may be trained by measuring the sensor array response to various biomarkers and adjusting the neural network parameters to minimize the error in the classification results, using known training techniques such as back-propagation.

In some embodiments of the invention, the system may capture baseline biometric readings of a user in a control iteration. The control iteration may involve immersing a user in a control mixed-reality environment that is designed not to provoke any emotional response from the user by default and to monitor the user's biometric information while the user is within the environment, thereby assessing a default emotional state. In some embodiments of the invention, the system may administer virtual content elements generically associated with certain emotions to the user in the control mixed-reality environment while measuring biometric information of the user, thereby preliminarily assessing which virtual content elements provoke which changes in the emotional state of the user.

The system may assess the emotional state of the user based on the biometric information by providing the biometric information to a machine learning model which may be trained to recognize patterns in the biometric information that correspond to different emotional states to produce a feature vector for each biosensor, where the feature vector represents a confidence level regarding the presence of each possible emotion based on the data from the associated biosensor. The system may then utilize a decision tree to process the feature vectors corresponding to all the biometric sensors present in the system to determine which emotion is most likely, based on the emotion with the highest collective confidence level; the system may then judge that emotion to comprise the current emotional state of the user. In some embodiments of the invention, the system may provide the same biometric information to multiple machine learning models to improve redundancy, reduce the effect of errors, and improve accuracy. In some embodiments of the invention, the system may determine multiple emotions to be most likely and may judge multiple emotions to comprise the current emotional state of the user. The system may initially train the machine learning model or models on generic relationships between a generic user's biometric information and a generic user's mental state based on statistical analysis which may be pre-provided, extracted from crowdsourced data, crawled from a repository, et cetera, and may adjust the relationships during operation based on the user's biometric information to better represent the connection between the biometric information and mental state of the individual user. The user's biometric information may be stored in a profile uniquely corresponding to that user. In some embodiments of the invention, the machine learning model or models may compare the biometric information gathered during the mixed-reality session against the default emotional state identified in a control iteration to better identify the user's emotional state. The system may perform the mental state assessment upon initialization of the mixed-reality experience, in response to biometric information being received from sensors, at regular intervals, et cetera.

In some embodiments of the invention, the biometric information is fed into a neural network for classification. The neural network may be, for example, a simple fully connected neural network with five sensor inputs, a hidden layer with three nodes, and two output nodes. An output may correspond to confidence in detecting a certain emotion; for example, output 1 and output 2 may correspond to joy and fear, respectively. The neural network inputs may be quantities sampled from sensor measurements such as measured features including voltages or readout currents measured and then digitized by analog-to-digital converters, or features extracted from the measurements using signal processing techniques. The extracted features may or may not have physical meanings. Examples of features with physical meanings may include output impedance, self-gain, subthreshold slope, et cetera. In at least one embodiment, the neural network computation is performed by an inference engine implemented as an application-specific integrated circuit (ASIC) which is either fabricated monolithically with the sensor chip or fabricated separately and integrated heterogeneously, for example bonded to the sensor chip.

In some embodiments of the invention, once a plurality of machine learning models have been initially trained, a manager network may be introduced to retrain the neural networks based on the cumulative output layer of each machine learning model. The manager network may take as input the emotional state predictions from the neural network or other machine learning models, which may comprise any number or combination of, for example, recurrent neural networks, convolutional neural networks, and long short-term memory networks. The manager network may be trained to minimize the error in the emotional state predictions. The output of the manager network may be a stream of retrained emotional state predictions that are used by the system to render the virtual environment in a way that is appropriate to the user's emotional state. Rendering the virtual environment in a way that is appropriate to the user's emotional state may, for instance, comprise rendering the emotional state to improve a user's joy, reduce a user's stress or anger or fear in situations where stress, anger and fear are not emotions that the mixed reality experience is intended to evoke, reduce a user's stress or anger or fear in situations where the user's stress, anger or fear are in excess of that which that the mixed reality experience is intended to evoke, et cetera.

In some embodiments of the invention, the system, and/or the manager network, may assign a weight to multiple different biosensors based on the value of sensor data from those sensors in accurately characterizing the mental state of the user. For example, the system may assign a higher weight on biosensor array classification than the body temperature classification for all or certain features based on determining that the biosensor array's classifications are more reliably accurate than the body temperature classifications. In another example, the system may assign a particularly high weight for stress classification by the biosensor array because biosensors are known to be particularly good at detecting cortisone. In some embodiments of the invention, the manager network learns these weights during training.

According to at least one embodiment, the profile, or user profile, may be a list of virtual content elements and the one or more emotions that each virtual content element evokes in a user, and potentially the magnitude to which the virtual content element evokes that emotion. The magnitude may be expressed as a number, which represents the amount to which the user's emotional state is affected by the virtual content in one or more contexts. The virtual content elements may be video, imagery, and/or audio elements that may be injected into a mixed reality session to influence the emotional state of the user. Virtual content elements may, for example, include uplifting text, music that makes the user feel happy or sad, colors or color schemes that make a user feel more agitated or more relaxed, cramped fonts or harsh dialogue that increase user anxiety, environmental effects such as rain that soothe a user, images that the user feels nostalgic for, et cetera. In some embodiments of the invention, virtual content elements may be characteristics of the mixed-reality environment or program itself, such as a difficulty level of a game, volume of audio, size and/or behavior of a simulated crowd around the user, behavior of simulated characters, et cetera. The profile may further comprise metadata associated with the virtual content elements providing identifying information about each virtual content element, such as for example the type of virtual content element (audio, video, gameplay, et cetera), the emotion or emotions evoked by the virtual content element in generic users and/or the user, the mixed reality programs or experiences associated with the virtual content element and/or within which the virtual content element could be injected, et cetera. The profile may further comprise the relationships between biometric sensor data gathered on the user and emotions of the user, as determined through operation of the system. For example, if the user has a particular habit of fidgeting when he is stressed, this behavior may be identified and represented as sensor data, and its relationship to stress in the user may be recorded in the user profile.

In some embodiments, virtual content elements may initially be selected for the profile from a list and/or repository based on the effect that a virtual content element has on a generic user as derived through statistical analysis, or the effect that a virtual content element has on a generic user with one or more matching interests or traits of the user; the system may then, for example by introducing the content element to the user in the control mixed-reality environment or in other mixed-reality environments, assess the particular effect that the virtual content element produces on the mental state of the user. According to at least one embodiment, the mapping of the virtual content elements to a generic user's mental state may be based on public historical responses which could be cataloged based on a larger population. Within certain scenarios, the use of crowdsourcing emotional responses may be utilized for certain types of content requirements. In some embodiments of the invention, virtual content elements may be associated with particular mixed reality programs or experiences; the system may select virtual content elements associated with a given mixed reality program or experience based on determining that the user owns, has access to, and/or is initializing an instance of that mixed reality program or experience. The system may also select virtual content elements to add to the profile as they are added or become available to the system. As the system operates over time, virtual content elements may become more tailored to the user's preferences, for example comprising the user's favorite songs, favorite color, favorite images, et cetera.

According to at least one embodiment, the system may, responsive to identifying that the emotional state of the user differs from the intended emotion of the mixed-reality experience, select one or more virtual content elements to add into the mixed reality environment. The system may identify an emotion that the mixed reality experience is intended to evoke in the user, and may compare this emotion against the emotional state of the user; if the emotions are not the same, the system may consult the profile to determine which virtual content elements are associated with the same emotion intended to be evoked by the mixed reality content. The system may then modify the mixed reality session with the selected virtual content by injecting the selected content into the mixed reality environment. The system may modify the mixed reality session by interfacing directly with the mixed-reality program hosting and/or executing the mixed-reality session, through for example an application programming interface, such that the system may provide or identify virtual content elements to inject into the mixed reality experience to the mixed-reality program, and instruct the mixed-reality program to add the virtual content elements to the mixed reality session at a desired place and time. The system may continue to monitor the user's emotional state, record the effect of the injected content element on the user's emotions and provide the effect as feedback to the machine learning model or models.

In some embodiments of the invention, an exemplary use case of the system may be illustrated in the following example:

Mirabelle puts on a biometric-sensor-equipped virtual reality headset and initiates a mixed-reality session comprising a mixed-reality experience emulating a roller coaster ride. The system begins collecting biometric data once the mixed-reality session is initiated and Mirabelle has opted-in to biometric data collection by communicating her assent through a prompt presented by the system via the VR headset's display. The system collects stress biomarkers using a biosensor array, body temperature through a thermal sensor, and speech data through a microphone, all integrated into the VR headset. The sensor data is fed into a neural network to produce feature vectors such as (joy, fear, stress), where the values of the feature vector represent the degrees of classification confidence for each possible emotion. The temperature reading from Mirabelle's headset produces the feature vector (0.3, 0.5, . . . 0.5) meaning that the neural network has 30% confidence that the emotion is (or includes) joy, 50% confidence that the emotion is (or includes) fear and 50% confidence that the emotion is (or includes) stress. This reading does not mean much by itself other than that fear, and stress are probably more likely than joy. However, the readings from the biosensor array pertaining to biomarkers produces the feature vector (0.6, 0.6, . . . 0.3), which means that joy and fear are more likely than stress. Finally, the microphones in the headset are picking up vocalizations from Mirabelle that produce feature vectors of (0.2, 0.6, . . . 0.6); accordingly, the system concludes that fear is probably the most likely (or dominant) emotion. Responsive to determining that the mixed-reality experience is a roller coaster ride and is therefore intended to evoke excitement but not fear, the system selects VR elements to reduce the fear-inducing nature of the roller coaster ride; the system slows down the roller coaster, brightens the colors, changes the music to be less intense and more relaxing, and reduces the height of the roller coaster. The system continues to monitor Mirabelle's emotions and finds that her joy has increased, and her fear has decreased; the system accordingly uploads the feedback to Mirabelle's profile and/or to the neural network to improve classification.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

The following described exemplary embodiments provide a system, method, and program product to gather biometric information on a user including biomarkers in the user's sweat, utilize the biometric information to identify a mental state of the user, and modify the user's mixed reality experience based on the identified mental state.

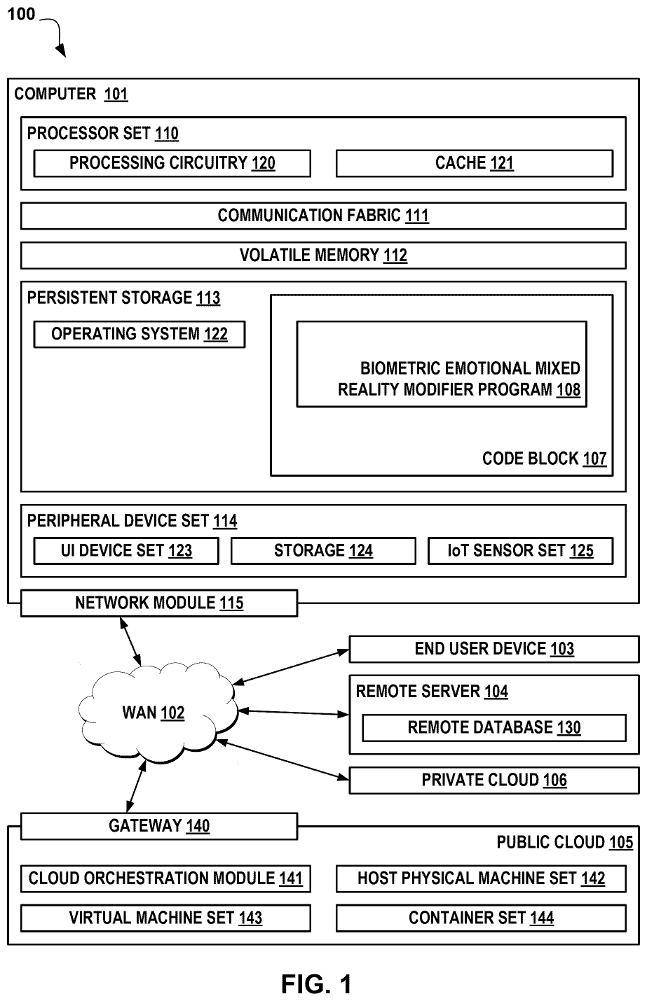

Referring now to FIG. 1, computing environment 100 comprises code block 107, which contains an example of an environment for the execution of at least some of the computer code involved in performing the inventive methods, such as biometric emotional MR modifier program 108. In addition to block 107, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 107, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 107 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface type operating systems that employ a kernel. The code included in block 107 typically includes at least some of the computer code involved in performing the inventive methods.

PERIPHERAL DEVICE SET 114 includes the set of peripheral devices of computer 101.

Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles, mixed reality headsets, and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications, including biometric sensors. For example, one sensor may be an infrared camera and another sensor may be a heart rate monitor.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

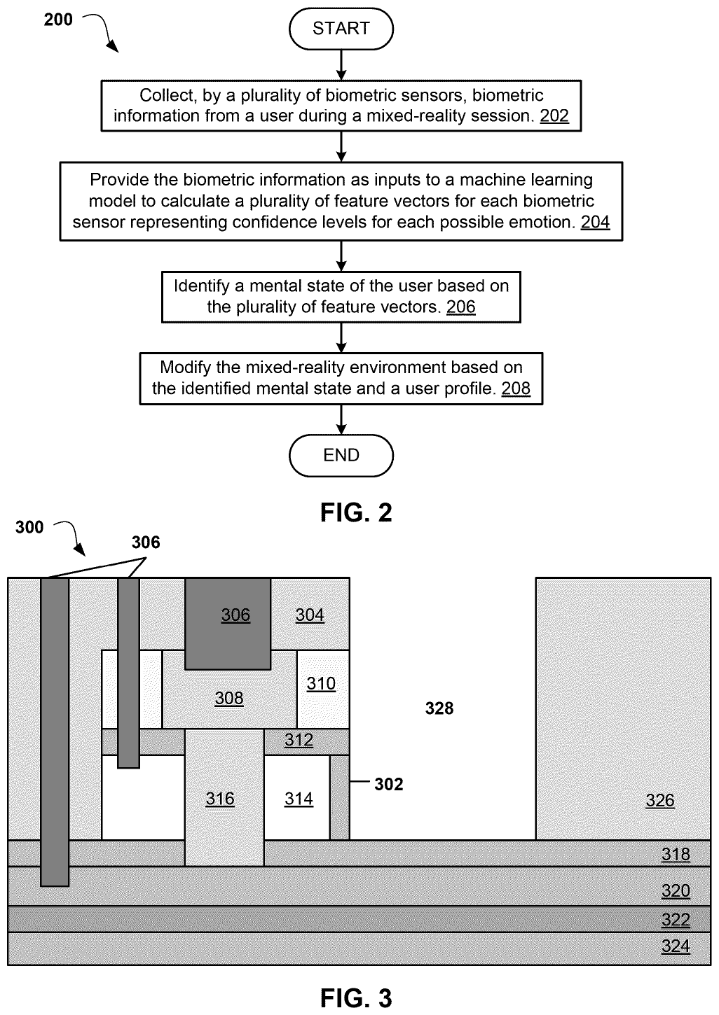

According to the present embodiment, the biometric emotional MR modifier program 108 may be a program enabled to gather biometric information on a user including biomarkers in the user's sweat, utilize the biometric information to identify a mental state of the user, and modify the user's mixed reality experience based on the identified mental state. The biometric emotional MR modifier program 108 may, when executed, cause the computing environment 100 to carry out a biometric emotional MR modifier method. The biometric emotional MR modifier method is explained in further detail below with respect to FIG. 2.

Referring now to FIG. 2, an operational flowchart illustrating a biometric emotional MR modifier process 200 is depicted according to at least one embodiment. At 202, the biometric emotional MR modifier program 108 collects, by a plurality of biometric sensors, biometric information from a user during a mixed-reality session. The biometric emotional MR modifier 108 may collect biometric information through one or more sensors which may be standalone devices or may be integrated into devices such as wearable vitality trackers, mixed reality devices, mobile devices, et cetera. Biometric information of a user may be collected continuously or at regular intervals, for example during a mixed reality session. In some embodiments of the invention, biometric data may be collected at points indicated by an author of a mixed-reality experience where a user's emotional state is intended to be at an expected baseline, such as the story beats of a narrative experience.

The biometric emotional MR modifier 108 may receive from a user explicit consent and permission for biometric emotional MR modifier 108 to collect biometric information on the user. The biometric emotional MR modifier 108 may prompt the user for such consent using a text and/or graphical prompt, for instance on the user's mobile device, which may detail the types, times, and contexts of biometric information collection that the biometric emotional MR modifier 108 would collect. In response to the prompt, the user may indicate either full or partial consent to biometric information collection. The biometric emotional MR modifier 108 may only collect biometric information that the user has consented to and may only use such collected biometric information for purposes communicated to the user.

At 204, the biometric emotional MR modifier program 108 provides the biometric information as inputs to a machine learning model to calculate a plurality of feature vectors for each biometric sensor representing confidence levels for each possible emotion. The biometric emotional MR modifier 108 may assess the emotional state of the user based on the biometric information by providing the biometric information to a machine learning model which may be trained to recognize patterns in the biometric information that correspond to different emotional states to produce a feature vector for each biosensor, where the feature vector represents a confidence level regarding the presence of each possible emotion based on the data from the associated biosensor. In some embodiments of the invention, the biometric emotional MR modifier 108 may provide the same biometric information to multiple machine learning models to improve redundancy, reduce the effect of errors, and improve accuracy. In some embodiments of the invention, the biometric emotional MR modifier 108 may assign weights to each biosensor comprising the biometric emotional MR modifier 108 based on the value of biometric information collected by that biosensor in accurately characterizing the mental state of the user.

At 206, the biometric emotional MR modifier program 108 identifies a mental state of the user based on the plurality of feature vectors. The biometric emotional MR modifier 108 may utilize a decision tree to process the feature vectors corresponding to all the biometric sensors present in the biometric emotional MR modifier 108 to determine which emotion is most likely, based on the emotion with the highest collective confidence level; the biometric emotional MR modifier 108 may then judge that emotion to comprise the current emotional state of the user. In some embodiments of the invention, the biometric emotional MR modifier 108 may determine multiple emotions to be most likely and may judge multiple emotions to comprise the current emotional state of the user. In some embodiments of the invention, the biometric emotional MR modifier 108 may determine which emotion is most likely based on the weights assigned to each sensor, considering more valuable biosensor data with higher weights to be more impactful in influencing the mental state decision.

At 208, the biometric emotional MR modifier 108 modifies the mixed-reality environment based on the identified mental state and a user profile. The biometric emotional MR modifier 108 may select one or more virtual content elements from a profile based on the biometric information. The biometric emotional MR modifier 108 may, responsive to identifying that the emotional state of the user differs from the intended emotion of the mixed-reality experience, select one or more virtual content elements to add into the mixed reality environment. The biometric emotional MR modifier 108 may identify an emotion that the mixed reality experience is intended to evoke in the user, and may compare this emotion against the emotional state of the user; if the emotions are not the same, the biometric emotional MR modifier 108 may consult the profile to determine which virtual content elements are associated with the same emotion intended to be evoked by the mixed reality content. The biometric emotional MR modifier 108 may then modify the mixed reality session with the selected virtual content by injecting the selected content into the mixed reality environment. The biometric emotional MR modifier 108 may modify the mixed reality session by interfacing directly with a mixed-reality program hosting and/or executing the mixed-reality session, through for example an application programming interface, such that the biometric emotional MR modifier 108 may provide or identify virtual content elements to inject into the mixed reality experience to the mixed-reality program, and instruct the mixed-reality program to add the virtual content elements to the mixed reality session at a desired place and time. The biometric emotional MR modifier 108 may continue to monitor the user's emotional state, record the effect of the injected content element on the user's emotions and provide the effect as feedback to the machine learning model or models.

Referring now to FIG. 3, an exemplary biosensor 300 for collecting biomarkers is depicted, according to at least one embodiment. Biosensor 300 is a lateral flow sensor with one sensing surface 302, which comprises a left ILD 304, which is pierced through by three contacts 306, which serve to electrically connect the layers of the biosensor 300. The rightmost contact 306 pierces through the ILD 304 into a high-doped, large-grain poly n+ collector 308, which is disposed within an oxide layer 310. The middle contact 306 pierces through the left ILD 304, through the oxide layer 310, through a top spacer 312, and into a doped base 314, which is also large grain poly. Disposed within the doped base 314 is an epitaxy 316. The leftmost contact 306 pierces through ILD 304, bottom spacer 318, and into n+− emitter layer 320, which may be a high-doped epitaxy. The n+− emitter layer 320 is disposed above an isolation layer 322, which is in turn disposed above a substrate 324, which may comprise silicon. Disposed on top of the bottom spacer 318 is right ILD 326; the space between left ILD 304 and right ILD 326 comprises sample trench 328. Samples of biomarker may be collected within sample trench 328 and analyzed using sensing surface 302.

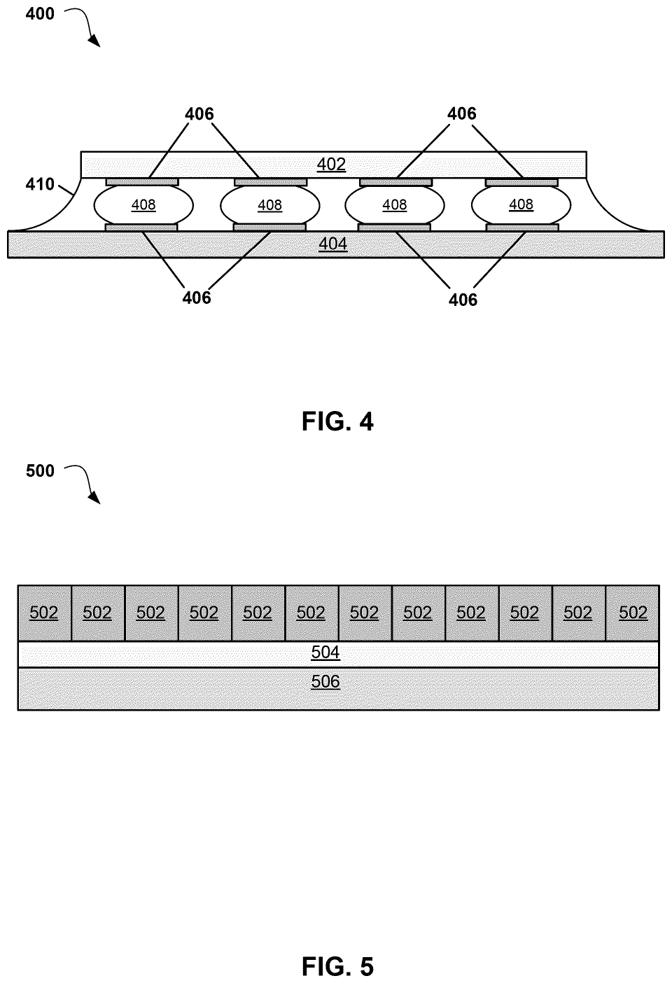

Referring now to FIG. 4, an exemplary biosensor array 400 for collecting biomarkers is depicted, according to at least one embodiment. Here, a sensor chip 402 is bonded, for example using flip-chip bonding, to a peripheral chip 404. The sensor chip 402 may comprise any number or combination of biosensors, including biosensor 300. The peripheral chip 404 may integrate additional functionalities, including memory, battery power, communication, processing, et cetera. The sensor chip 402 may be bonded to the peripheral chip 404 by a series of pairs of connector plates 406 disposed on opposing surfaces of the sensor chip 402 and the peripheral chip 404, connected by solder balls 408. The space between the plates 406 and the solder balls 408 may be filled with underfill 410.

Referring now to FIG. 5, an exemplary biosensor array 500 for collecting biomarkers is depicted, according to at least one embodiment. Biosensor array 500 may comprise any number and combination of biosensors 502, including, for example, biosensor 300, disposed upon a bonding layer 504 which is in turn disposed upon a flexible base layer 506 which comprises analyzing circuitry and wireless communications devices. Though the biosensor array 500 may comprise any number and combination of biosensors 502, one skilled in the art would understand that more biosensors 502 provide more accuracy, and that the array 500 should comprise more than two biosensors 502. Because base layer 506 is flexible, the biosensor array 500 is flexible, and may be worn like a patch on the skin.

It may be appreciated that FIGS. 2-5 provides only illustrations of individual implementations and do not imply any limitations with regard to how different embodiments may be implemented. Many modifications to the depicted environments may be made based on design and implementation requirements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for biometric mixed-reality emotional modification, the method comprising:
collecting, by a plurality of biosensors, biometric information on a user during a mixed-reality session, wherein the biometric information comprises biomarkers;
identifying, by one or more machine learning models, a mental state of the user based on the biometric information; and
responsive to determining that the mental state does not match an intended emotion associated with a mixed-reality experience, modifying the mixed-reality experience with one or more virtual content elements.

2. The method of claim 1, wherein the virtual content elements are selected from a profile based on a predicted emotional effect the virtual content element will have on the emotional state.

3. The method of claim 1, wherein the identifying further comprises: generating a feature vectors associated with the biosensors; and determining the mental state based on determining one or more dominant emotions of the user based on the feature vectors.

4. The method of claim 1, wherein the identifying is based on weights associated with the plurality of biosensors representing a value of the biosensors' biometric data in identifying the mental state.

5. The method of claim 1, wherein a plurality of biomarker biosensors of the plurality of biosensors comprise a densely packed array wherein a sensing surface of the biomarker biosensors are differently functionalized to produce correlated responses to different biomarkers.

6. The method of claim 1, further comprising: retraining, by a manager network, the one or more machine learning models based on the cumulative output layer of each machine learning model.

7. The method of claim 1, wherein the biometric information further comprises body temperature and speech information.

8. A computer system for biometric mixed-reality emotional modification, the computer system comprising:
one or more processors, one or more computer-readable memories, one or more mixed-reality devices, one or more sensors, one or more computer-readable tangible storage medium, and program instructions stored on at least one of the one or more tangible storage medium for execution by at least one of the one or more processors via at least one of the one or more memories, wherein the computer system is capable of performing a method comprising:
collecting, by a plurality of biosensors, biometric information on a user during a mixed-reality session, wherein the biometric information comprises biomarkers;
identifying, by one or more machine learning models, a mental state of the user based on the biometric information; and
responsive to determining that the mental state does not match an intended emotion associated with a mixed-reality experience, modifying the mixed-reality experience with one or more virtual content elements.

9. The computer system of claim 8, wherein the virtual content elements are selected from a profile based on a predicted emotional effect the virtual content element will have on the emotional state.

10. The computer system of claim 8, wherein the identifying further comprises: generating a feature vectors associated with the biosensors; and determining the mental state based on determining one or more dominant emotions of the user based on the feature vectors.

11. The computer system of claim 8, wherein the identifying is based on weights associated with the plurality of biosensors representing a value of the biosensors' biometric data in identifying the mental state.

12. The computer system of claim 8, wherein a plurality of biomarker biosensors of the plurality of biosensors comprise a densely packed array wherein a sensing surface of the biomarker biosensors are differently functionalized to produce correlated responses to different biomarkers.

13. The computer system of claim 8, further comprising: retraining, by a manager network, the one or more machine learning models based on the cumulative output layer of each machine learning model.

14. The computer system of claim 8, wherein the biometric information further comprises body temperature and speech information.

15. A computer program product for biometric mixed-reality emotional modification, the computer program product comprising:
one or more computer-readable tangible storage media and program instructions stored on at least one of the one or more tangible storage media, the program instructions executable by a processor to cause the processor to perform a method comprising:
collecting, by a plurality of biosensors, biometric information on a user during a mixed-reality session, wherein the biometric information comprises biomarkers;
identifying, by one or more machine learning models, a mental state of the user based on the biometric information; and
responsive to determining that the mental state does not match an intended emotion associated with a mixed-reality experience, modifying the mixed-reality experience with one or more virtual content elements.

16. The computer program product of claim 15, wherein the virtual content elements are selected from a profile based on a predicted emotional effect the virtual content element will have on the emotional state.

17. The computer program product of claim 15, wherein the identifying further comprises: generating a feature vectors associated with the biosensors; and determining the mental state based on determining one or more dominant emotions of the user based on the feature vectors.

18. The computer program product of claim 15, wherein the identifying is based on weights associated with the plurality of biosensors representing a value of the biosensors' biometric data in identifying the mental state.

19. The computer program product of claim 15, wherein a plurality of biomarker biosensors of the plurality of biosensors comprise a densely packed array wherein a sensing surface of the biomarker biosensors are differently functionalized to produce correlated responses to different biomarkers.

20. The computer program product of claim 15, further comprising: retraining, by a manager network, the one or more machine learning models based on the cumulative output layer of each machine learning model.
